(19) United States
(12) Patent Application Publication
(10) Pub. No.: US 2012/0116350 A1
(43) Pub. Date: May 10, 2012

(54) TRANSLATION DILATOR AND STAND ALONE VASCULAR GUIDE CATHETER

(75) Inventors: Brian M. Strauss, Trabuco Canyon, CA (US); Lynn Shimada, Orange, CA (US); Serefino Torres, Mission Viejo, CA (US); Jay A. Lenker, Laguna Beach, CA (US)

(73) Assignee: REVERSE MEDICAL CORPORATION, Irvine, CA (US)

(21) Appl. No.: 13/178,381

(22) Filed: Jul. 7, 2011

Related U.S. Application Data
(60) Provisional application No. 61/362,208, filed on Jul. 7, 2010.

(51) Int. Cl. A61B 17/22; A61M 25/14; A61M 29/02

(52) U.S. Cl. 604/508; 604/103.07; 606/200

(57) ABSTRACT

Systems and methods for delivering implantable devices, catheters, or substances in or near and/or restoring flow through body lumens, such as blood vessel lumens are described. A catheter having a proximal portion of a first diameter and a distal portion of a second diameter (smaller than the first diameter) is advanced into a body lumen. The distal portion of the catheter is caused to expand to a diameter that is larger than the second diameter but no larger than the first diameter. A working device is then advanced out of the distal end of the catheter and used to remove obstructive matter, deliver an implantable device or substance and/or restore flow. The distal portion can be reduced in diameter prior to removal from the body. A stand alone, guide catheter is also disclosed possessing high resistance to kinking even with a very thin wall.

### RELATED APPLICATION

This patent application claims priority to U.S. Provisional Patent Application No. 61/362,208 filed Jul. 7, 2010, the entire disclosure of which is expressly incorporated herein by reference.

### FIELD OF THE INVENTION

The present invention relates generally to medical devices and methods and more particularly to catheter-based systems and methods useable for accessing, diagnosing, or treating defects in blood vessels, such as blood vessels of the brain.

### BACKGROUND OF THE INVENTION

Stroke is a common cause of death in the United States and disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to rupture of an aneurysm or arteriovenous malformation (AVM), causing bleeding into the brain tissue and resultant infarction of brain tissue. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs, which resolve completely within 1 hour, the term transient ischemic attack (TIA) is used. Etiologically, TIA and ischemic stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stroke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mmHg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Catheter-based thrombectomy devices, foreign body retrieval systems, or the like can be used to engage and retrieve emboli, which are found to be the source of neurological deficit. Although neurointerventional devices and procedures have advanced, there remains a need for expeditious restoration of distal flow to blocked, or stenotic, cerebrovascular vessels, which can lead to severe neurological deficit or patient death.

Catheter-based systems for treating certain cerebrovascular disorders rely on various therapies including delivery of hardenable polymers and other embolic agents to treat arteriovenous malformations (AVM). Catheter-based delivery systems can deploy thrombogenic coils, aneurysm occluders, stents, neck bridges, and other devices to treat cerebrovascular aneurysms.

Guide catheters are often used to direct catheter-based systems for treating hemorrhagic or ischaemic stroke. However, current guide catheters cannot be advanced near the treatment site if such treatment site is within the circle of Willis. The ability to reach into the deep cerebrovasculature will allow therapeutic microcatheters to be better controlled and provide for more efficacious stroke and aneurysm therapy.

New devices and methods are thus needed in treating vasculature occlusions or bleeding disorders in the body, including patients with acute ischemic stroke and occlusive cerebrovascular disease, in treating symptomatic patients with embolization or hemodynamic compromise, or in stroke prevention, e.g., patients with incidental finding of asymptomatic carotid lesion, which improve a patient's neurological function and quality of life without causing significant side effect, and can thus also be used in patients with contraindication to the use of t-PA.

### SUMMARY OF THE INVENTIONS

In accordance with one aspect of the present invention, there is provided a guide catheter device having a non-radially expandable distal portion (suitable for cerebrovascular access) comprising a composite structure of an inner liner, an outer polymer layer, and a reinforcement, wherein the guide catheter comprises at least two regions of flexibility determined, in part, or in whole, by the construction of the reinforcement in each of the two regions of flexibility.

Further in accordance with the present invention, there is provided a guide catheter device which comprises: (a) a non-diametrically expandable proximal tubing segment comprising an axially elongate tube having a proximal end, a distal end, and a lumen extending therethrough, the flexibility of said proximal tubing segment being greater at its distal end than at its proximal end; (b) a diametrically expandable distal segment affixed to the distal end of the proximal tubing segment, the distal segment responsive to expand diametrically upon axial movement of a hollow central dilator into or out of the distal segment; and (c) a hollow central dilator comprising a composite structure of an inner liner, an outer polymer layer, and a reinforcement, wherein the hollow central dilator comprises at least two regions of flexibility determined, in part, or in whole, by the construction of the reinforcement in each of the two regions of flexibility. A hub may be attached to the proximal end of the proximal tubing segment and such hub may comprise at least one hemostasis valve and at least one access port.

Further in accordance with the invention, there is provided a system useable for performing a therapeutic or diagnostic task at a location within the body of a human or animal subject, such system comprising a) catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion and b) a working device that is advanceable though the lumen of the catheter and out of its distal opening at least when the distal portion of the catheter is in is second configuration, said working device being useable to perform the therapeutic or diagnostic task. Examples of the types of working devices that may be used in this system include but are but are not limited to; i) devices for removing thrombus or other obstructive matter from body lumens, ii) flow restoration devices useable to facilitate flow of a fluid though or around an obstruction within a body lumen and iii) devices for deploying or delivering implants (e.g., implantable occlusion coils or implantable embolic devices). Non-limiting examples of catheters having expandable distal portions which may be used in accordance with this invention include that summarized in preceding Paragraph Nos. 0013 and 0014 as well as that described in United States Patent Application Publication No. US/2010/0114017, the entire disclosure of which is expressly incorporated herein by reference.

Further in accordance with the invention, there is provided a method for performing a therapeutic or diagnostic task at a location within the body of a human or animal subject, such method comprising the steps of: a) inserting into the subject's body a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the distal end opening in a desired body lumen while the distal portion of the catheter is in its first configuration; c) causing the distal portion of the catheter to transition to its second configuration; d) advancing a working device though the lumen of the catheter and out of its distal opening; and, using the working device to perform the therapeutic or diagnostic task. Examples of the types of working devices that may be used in this method include but are but are not limited to; devices for removing thrombus or other obstructive matter from body lumens, flow restoration devices useable to restore blood flow though an obstructed body lumen and devices for delivering implants (e.g., implantable occlusion coils or embolic devices).

Still further in accordance with the invention there is provided a method for removing obstructive matter from a body lumen, such method comprising the steps of: a) inserting a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the catheter, while in the first configuration, such that its distal end opening is within a body lumen; c) causing the catheter to transition from the first configuration to the second configuration; d) moving obstructive matter through the distal end opening and into the lumen of the catheter; and e) removing the catheter along with the obstructive matter that has been moved into the lumen of the catheter. In some embodiments, negative pressure may be applied through the lumen of the catheter to aspirate obstructive matter through the distal end opening and into the lumen of the catheter. In some embodiments Step D of the method may comprise advancing an obstructive matter moving device (e.g., an embolectomy device) from the catheter and using the obstructive matter-moving device to move obstructive matter through the distal end opening and into the lumen of the catheter.

Still further in accordance with the present invention, there is provided a method for increasing flow of a body fluid through an obstructed body lumen, such method comprising the steps of: a) inserting a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the catheter, while in the first configuration, such that its distal end opening is within a body lumen; c) causing the catheter to transition from the first configuration to the second configuration; and d) using the catheter to deliver a treatment that restores or improving flow of a body fluid through an obstructed body lumen. In some embodiments, the treatment delivered may comprise the delivery of a therapeutic substance (e.g., a thrombolytic agent) of a type and in an amount that is effective to improve flow of body fluid through the body lumen. In some embodiments, the treatment delivered may comprise use of a device that canalizes or compresses obstructive matter in a manner that improves flow of body fluid through or around the obstructive matter.

In other embodiments, a guide catheter is provided which does not transition from a first, smaller diameter to a second, larger diameter in the distal region. However, the guide catheter comprises construction that provides for high flexibility, high torqueability, high column strength, high resistance to kinking, excellent circularity, and very thin wall thickness. The guide catheter is constructed of a thermoplastic outer layer, a stainless steel or nitinol braid or coil, and a fluoropolymer liner. The guide catheter is fabricated using a co-extrusion or re-flow process in which the outer thermoplastic layer is melted and forms through the braid or coil to adhere, at least mechanically, to the inner liner layer.

The guide catheter composite construction comprises, in an embodiment, a 0.001-inch wall thickness polytetrafluoroethylene (PTFE) liner. A snake cut nitinol tube is terminated approximately 5 or more centimeters from the distal end of the catheter. The distal remainder of the guide catheter comprises a 0.0025 thick by 0.008 wide nitinol coil wound around the outside of the PTF liner. The gap between the windings is about 0.004 inches or 50% of the coil element width. The proximal end of the coil is laser welded to the distal end of the snake cut nitinol tube. Flexobond urethane adhesive is applied to the coil. Approximately 20 cm of polyolefin heat shrink tubing U4-140-CLR, from Cobalt Polymers is used to embed the coil distal end and extending over the snake cut or laser cut proximal end. A liner of 0.001 thick PET heat shrink tubing is applied over the snake cut or laser cut tubing. The polyolefin outer layer is fused and compressed over the coil such that substantially none of the polyolefin extends to form a layer on the exterior of the coil but rather extends between the coils and is fused to the PTFE liner. Alternatively, the polyolefin outer layer can comprise a thin web over the coil but the thickness is minimized to minimize the wall thickness of the structure.

For purposes of summarizing the invention, certain aspects, embodiments, variations, details, elements, examples, advantages, and novel features of the inventions are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following detailed description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate some but not all embodiments or examples of the invention and do not limit the scope of the claimed inventions in any way. Throughout the drawings, reference numbers are re-used to indicate correspondence between referenced elements.

FIG. 1 illustrates a breakaway view of the transition region near the distal end of a guide catheter or guide catheter dilator, wherein a coil-type proximal end reinforcement is welded to a coil-type distal end reinforcement, according to an embodiment of the invention;

FIG. 2 illustrates a breakaway view of the transition region near the distal end of a guide catheter or guide catheter dilator, wherein a snake-cut proximal end reinforcement is welded to a coil-type distal end reinforcement, according to an embodiment of the invention;

FIG. 3 illustrates the full length of a guide catheter or guide catheter dilator, wherein a snake-cut proximal end reinforcement is welded to a coil-type distal end reinforcement, according to an embodiment of the invention; and

FIG. 4 illustrates a breakaway view of the transition region near the distal end of a guide catheter or guide catheter dilator, wherein a snake-cut proximal end reinforcement is welded to a coil-type distal end reinforcement, the coil distal end reinforcement being shown in sectional view, according to an embodiment of the invention.

### DETAILED DESCRIPTION

The inventions disclosed herein may be embodied in other specific forms without departing from its spirit or essential characteristics. The described embodiments are to be considered in all respects only as illustrative and not restrictive. The scope of the inventions is therefore indicated by the appended claims rather than the foregoing description. All changes that come within the meaning and range of equivalency of the claims are to be embraced within their scope.

As used herein, the terms proximal and distal refer to a direction or a position along a longitudinal axis of a catheter or medical instrument. Proximal refers to the end of the catheter or medical instrument closer to the operator, while distal refers to the end of the catheter or medical instrument closer to the patient. For example, a first point is proximal to a second point if it is closer to the operator end of the catheter or medical instrument than the second point. The measurement term French, abbreviated Fr or F, is defined as three times the diameter of a device as measured in mm. Thus, a 3 mm diameter catheter is 9 French in diameter.

There is provided in accordance with one aspect of the present invention, a method for accessing regions of the vasculature through tortuous anatomy. Such vasculature includes the cerebrovasculature wherein access to the circle of Willis and beyond is exceedingly difficult due to the carotid siphon or vertebral artery anatomy that must be traversed to reach such locations. The method comprises the steps of providing a catheter having a proximal end, a distal end, an expandable distal section having a distal port, an aspiration lumen communicating with the port, and an axially movable support or dilator. The distal end of the catheter is inserted into the artery, and the support is distally advanced to expand the distal section. Negative pressure can be applied to the aspiration port, to draw the thromboembolic material into the distal section. Catheters and other instrumentation can be inserted through the movable support once it is placed within the vasculature. The movable support can be used, by itself, without the outer expandable sheath to gain access to locations where flexibility, kink resistance, torqueability, and column strength are required.

Typical arteries may be, among other examples, the common carotid artery, the internal carotid artery, the carotid siphon, the circle of Willis, etc. Alternatively, the artery may be the middle cerebral artery or the anterior cerebral artery, or elsewhere in the brain.

The method may additionally comprise the steps of introducing oxygenated medium into the artery through the aspiration lumen, or infusing pharmaceutical agent into the artery through the aspiration lumen. The pharmaceutical agent may be a vasodilator such as nifedipine or nitroprusside. The pharmaceutical agent may alternatively comprise t-PA. The thromboembolic material may be located using intravascular ultrasound, or carotid Doppler imaging techniques.

In accordance with another aspect of the present invention, there is provided an intracranial aspiration catheter. The catheter comprises an elongate flexible tubular body, having a proximal end, a distal end, and an aspiration lumen extending therethrough. The aspiration lumen in a distal section of the body is movable between a first, reduced inside diameter for transluminal navigation and a second, enlarged inside diameter for aspirating material. A support is provided, for controllably supporting the aspiration lumen against collapse when in the second diameter. A control is provided on the proximal end of the catheter for controlling the support. In one implementation, the support comprises a spiral element such as a spring coil. The support may be axially movable, such as between a proximal position when the distal section is in the low cross sectional configuration, and a distal position in which the distal section is enlarged, and supported against collapse under aspiration. Alternatively, the support is activated by rotating a first end of the support relative to a second end of the support.

The aspiration lumen may be defined within a tubular wall having a plurality of folds therein, when the aspiration lumen is in the first inside diameter configuration. Alternatively, the aspiration lumen may be defined within a wall made from a stretchable material.

In accordance with another aspect of the present invention, there is provided a method of establishing a flow path through a catheter, positioned across a non-linear segment of vasculature. The method comprises the steps of transluminally navigating an enlargeable tubular wall through a non-linear segment of vasculature, and manipulating a support within a tubular wall to enlarge the inside diameter of the tubular wall to create a flow path across the non-linear segment. The manipulating step may comprise distally advancing a tubular support structure within the tubular wall. In one implementation, the method comprises distally advancing a coil within the tubular wall.

In accordance with a further aspect of the present invention, there is provided a method of aspirating material. The method comprises the steps of transluminally advancing a catheter to the site of an obstruction, the catheter having an aspiration lumen therein. A support is moved within the aspiration lumen, and, thereafter, material is aspirated from the obstruction through the aspiration lumen.

In accordance with another aspect of the present invention, there is provided an intracranial aspiration catheter. The catheter comprises an elongate flexible tubular body, having a proximal end, a distal end, and an aspiration lumen extending therethrough. The distal section on the body is movable between a first, reduced inside diameter for transluminal navigation, and a second, enlarged inside diameter for aspirating material. A support is axially movable between a proximal position when the aspiration lumen is in the first diameter, and a distal position for supporting the aspiration lumen against collapse when in the second diameter.

In one implementation, the support comprises a coil. The distal section may have a length of no greater than about 30 cm, in certain embodiments a length of no greater than about 20 cm, and often within the range of from about 5 cm to about 15 cm.

In certain embodiments, the expandable aspiration catheter can serve as an expandable guide catheter for placement of the micro-catheter. The expandable guide catheter is advanced to a target region in cooperation with a guidewire to allow for steering and manipulation through the vasculature. In an exemplary procedure, the guidewire and expandable guide catheter are introduced into the vasculature at a site within a femoral or iliac artery. Using a Seldinger technique, or other percutaneous procedure, a hollow 18-Gauge needle can be introduced into a femoral artery via percutaneous procedure. A guidewire is next advanced through the hollow needle and into the arterial tree. The hollow needle is next removed and a catheter introducer is advanced into the arterial tree. The expandable guide catheter is next advanced through the catheter introducer either through the same guidewire or through a larger guidewire suitable for aortic traverse. The expandable guide catheter, in its radially collapsed configuration, is advanced through the aortic arch, into a carotid artery, through the carotid siphon and into a region proximate the circle of Willis. The distal end of the expandable guide catheter is next expanded by advancing an internal element distally to force the distal end radially outward and maintain an enlarged diameter inner lumen. The expandable guide catheter can provide a very small diameter, flexible catheter that is easily inserted through tortuous anatomy such as the carotid siphon or the vertebral and basilar arteries. Once properly placed, the expandable guide catheter can be diametrically expanded to generate a lumen larger than would be possible with a standard, non-expandable catheter. In addition, the expanded guide catheter can partially or completely straighten out the tortuous vasculature to allow passage of larger diameter, less flexible microcatheters suitable for advanced therapeutic or diagnostic purposes. The expanded guide catheter can serve as an aspiration device and as a shield for retrieval of debris, thrombus, or other material from the vasculature.

The disclosure herein is directed at apparatus suitable to be the central axially movable (translation) dilator for the radially expandable guide catheter or as the design for a free-standing guide catheter. The guide catheter is preferably terminated, at its proximal end, with a hemostasis valve and optionally with a connector offering multiple access ports, each of which can be valved or be terminated with a stopcock, etc.

There is disclosed a guide catheter or translation dilator 100 in accordance with one aspect of the present invention. Although primarily described in the context of a an expandable distal segment aspiration catheter with a single central lumen, catheters of the present invention can readily be modified to incorporate additional structures, such as permanent or removable column strength enhancing mandrels, two or more lumen such as to permit drug or irrigant infusion or radiation delivery or to supply inflation media to an inflatable balloon, or combinations of these features, as will be readily apparent to one of skill in the art in view of the disclosure herein. In addition, the present invention will be described primarily in the context of removing obstructive material from remote vasculature in the brain.

The catheters disclosed herein may readily be adapted for use throughout the body wherever it may be desirable to introduce a low profile catheter and then provided a relatively large diameter aspiration or supported working channel. For example, low diameter catheter shafts in accordance with the present invention may be dimensioned for use throughout the coronary and peripheral vasculature, the gastrointestinal tract, the urethra, ureters, Fallopian tubes and other lumens and potential lumens, as well. The expandable lumen structure of the present invention may also be used as a minimally invasive percutaneous tissue tract expander, such as for diagnostic or therapeutic access to a solid tissue target (e.g., breast biopsy or tissue excision).

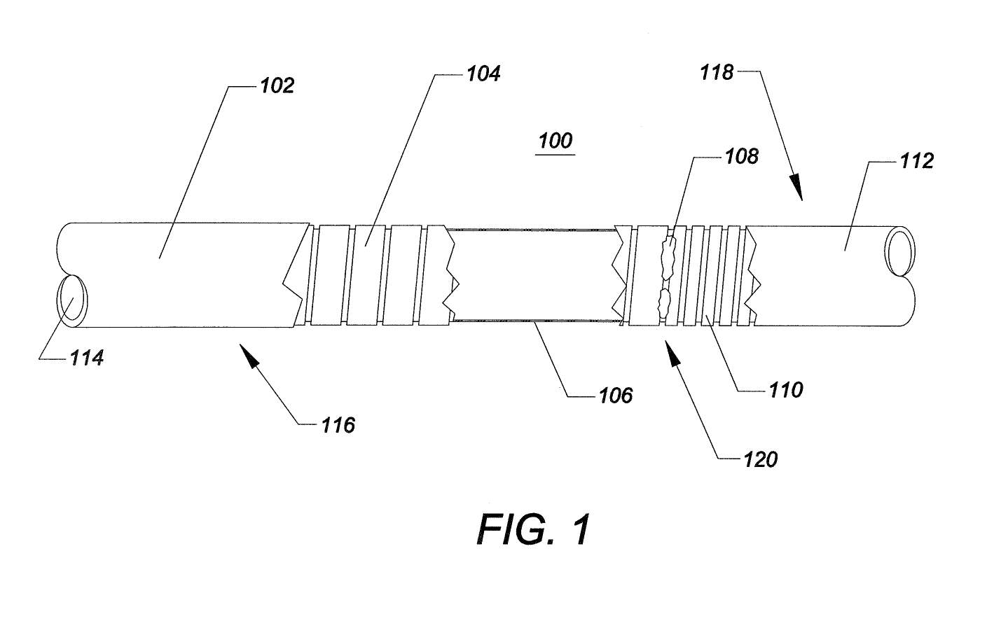

FIG. 1 illustrates a side partial breakaway view of a translation dilator or stand-alone guide catheter 100 near a transition zone 120. The guide catheter 100 comprises a proximal section 116, a distal section 118, and a lumen 114 extending therethrough. The guide catheter 100 further comprises an inner sleeve 106, a proximal reinforcement 104, a proximal outer layer 102, a distal reinforcement 110, a distal outer layer 112, and a transition weld 108.

Referring to FIG. 1, the inner sleeve 106 is surrounded by the proximal reinforcement 104, which can be a metallic coil, braid, snake cut, stent, or other reinforcing structure. The proximal outer layer 102 is compressed with heat and pressure over the proximal reinforcement 104 such that it extrudes through the spaces between the elements of the reinforcement 104 and bonds, at least mechanically, with the inner sleeve 106. The central lumen 114 is surrounded by the inner sleeve 106. The distal reinforcement 110 is embedded within the outer distal layer 112 such that the outer distal layer 112 surrounds and encompasses the distal reinforcement 110 and extends through the spaces between the coils to bond, at least mechanically, with the inner sleeve 106. The distal section 118 can range in length from about 5 cm to about 30 cm, depending on the distance of tortuous anatomy that needs to be traversed by the device.

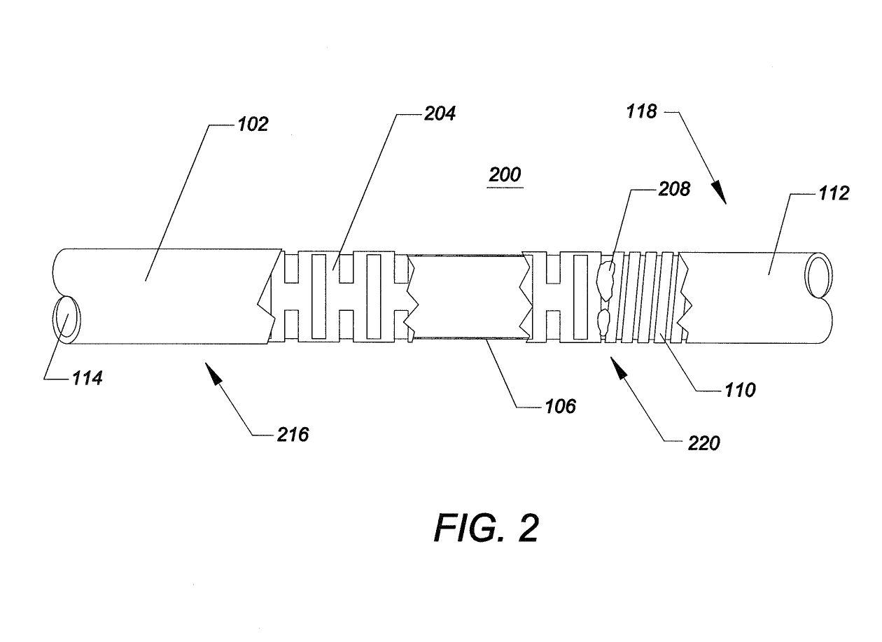

FIG. 2 illustrates a side partial breakaway view of a translation dilator or stand-alone guide catheter 200 near a transition zone 220. The guide catheter 200 comprises a proximal section 216, the distal section 118, and a lumen 114 extending therethrough. The guide catheter 200 further comprises the inner sleeve 106, a proximal reinforcement 204, the proximal outer layer 102, the distal reinforcement 110, the distal outer layer 112, and the transition weld 208.

Referring to FIG. 2, the transition weld 208 affixes the distal end of the proximal reinforcement 204 to the proximal end of the distal reinforcement 110. The transition weld 208 can be achieved using a laser welder, a plasma welder, a micro-arc welder, and the like. The polymers comprising the proximal outer layer 102 and the distal outer layer 112 are heat formed together in the region of the transition 220 using heat shrink tubing or other compression methodology and heat, such as that generated by a heated air flow source, radiant heat source, induction heater, radiofrequency heater, or the like. The proximal reinforcement 204 is a metallic structure etched with perforations such that it forms a structure known as a snake cut. The perforations in the proximal reinforcement 204 are disposed such that distributed flexibility can be generated in a metal tube that is snake cut. The perforations can cause the flexibility to be substantially evenly distributed, or directed along a specific axis. The metal used in the proximal reinforcement can be nitinol, stainless steel, cobalt-nickel alloy, titanium, or the like.

The proximal reinforcements 104, 204 can beneficially be created such that the reinforcement becomes more flexible moving distally. This increasing flexibility can be created at in discreet regions or can be continuously changed. Increasing flexibility can, for example be created by using a snake cut proximal reinforcement 204 in the most proximal regions, transitioning to a coil reinforcement 104 in intermediate regions, transitioning to the wire wound distal reinforcement 110 in the most distal part of the guide catheter 100 or 200.

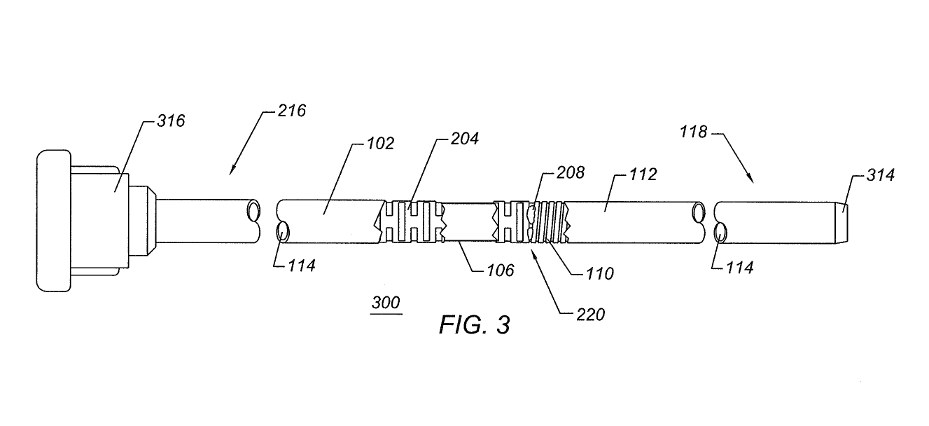

FIG. 3 illustrates a side partial breakaway view of a translation dilator or stand-alone guide catheter 300. The guide catheter 300 comprises the proximal section 216, the distal section 118, the transition zone 220, the inner lumen 114, a proximal hub 316, and a distal bevel 314. The guide catheter 300 further comprises the inner sleeve 106, a proximal reinforcement 204, the proximal outer layer 102, the distal reinforcement 110, the distal outer layer 112, and the transition weld 208.

Referring to FIG. 3, the proximal hub 316 is affixed to the proximal end of the proximal section 216 by welding, adhesives, mechanical fasteners, or the like. The proximal hub 316 can comprise hemostasis valves, sideports, purge lines, and the like. The proximal hub 316 can comprise a plurality of sideports for insertion of guidewires, catheters, implantable devices, pharmaceutical agents, and the like. Each sideport is beneficially terminated with a hemostasis valve, stopcock, or the like, to prevent blood from escaping from the proximal end of the guide catheter 300. The distal bevel 314 is integral to the distal section 118 and facilitates advancement of the guide catheter 300 through an expandable outer sheath (not shown), or through the vasculature, itself. The distal bevel 314 is illustrated with a ten degree taper but could taper at angles between about 5 degrees to about 45 degrees. The proximal hub 316 can comprise wings for improved torque handling and luer connections to facilitate attachment of lines to the hub 316.

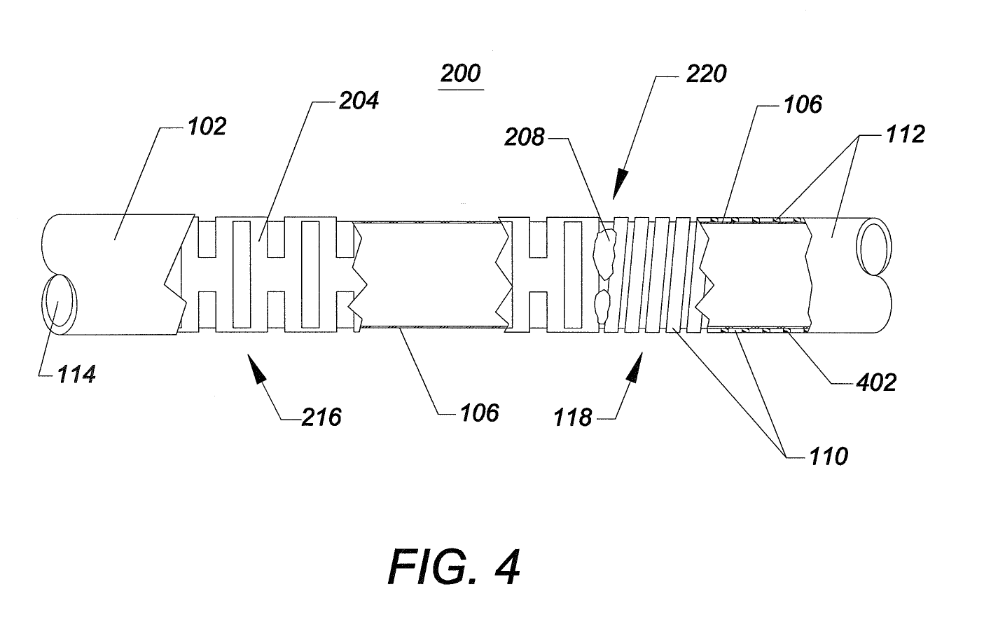

FIG. 4 illustrates a side partial breakaway view of the translation dilator or stand-alone guide catheter 200 near the transition zone 220. The guide catheter 200 comprises a proximal section 216, the distal section 118, and a lumen 114 extending therethrough. The guide catheter 200 further comprises the inner sleeve 106, a proximal reinforcement 204, the proximal outer layer 102, the distal reinforcement 110, the distal outer layer 112, and the transition weld 208. The distal region 118 is also illustrated in cross-section exposing the gaps 402 between the coil elements 110 for observation.

The polymer outer layer 112 in the distal region 118 is compressed over the distal reinforcement 110 such that little or no material remains outside of the coils of the distal reinforcement 110. Said little or no material thickness can range from about 0 to about 0.010 inches and preferably between about 0 and 0.005 inches, and most preferably between about 0 and 0.001 inches. The dimensions of the coil elements in the distal reinforcement 110, in a preferred embodiment, are about 0.0025 inches thick by about 0.008 inches wide. The spacing between the coils of the distal reinforcement 110 is about 0.004 inches or about 50% of the coil element width. The outer layer 112 can be fabricated from polyolefin heat shrink tubing, polyolefin, or the like. Material denoted as U4-140-CLR from Cobalt Polymers is a suitable polyolefin for the outer distal layer 112 and also for outer proximal layer 102. The wall thickness of the inner sleeve 106 is about 0.001 inches. The material of the inner sleeve 106 can be PTFE, PFA, FEP, or other fluoropolymer. It is beneficial that the inner sleeve 106, forming the wall of the inner lumen 114, is very smooth and lubricious. An exemplary design comprises an inner diameter of about 0.070 inches, and an outside diameter of about 0.078 to about 0.080 inches.

The catheter 100, 200, 300 generally comprises an elongate tubular body extending between a proximal end and a distal functional end 314. The length of the tubular body depends upon the desired application. For example, lengths in the area of from about 90 cm to about 140 cm or more are typical for use in femoral access percutaneous transluminal coronary applications. Intracranial or other applications may call for a different catheter shaft length depending upon the vascular access site, as will be understood in the art.

In certain embodiments where the structure is used as an axially movable translation dilator, the tubular body through which it is slidably disposed is divided into at least a fixed diameter proximal section and an adjustable diameter distal section separated by a transition, discussed infra. Alternatively, the adjustable diameter feature of distal section can extend the entire length of the catheter from the manifold or other proximal connector to distal tip, as will become apparent from the disclosure herein.

The proximal end of the catheter is additionally provided with a manifold having one or more access ports as is known in the art. Generally, the manifold is provided with a guidewire port in an over-the-wire construction, an aspiration port, and a catheter insertion port. One or more of these features can be embodied within a single port. Alternatively, the aspiration port may be omitted if the procedure involves removal of the guidewire proximally from the guidewire port following placement of the aspiration catheter, and aspiration through the guidewire port. Additional access ports may be provided as needed, depending upon the functional capabilities of the catheter. The manifold may be injection molded from any of a variety of medical grade plastics, or formed in accordance with other techniques known in the art.

The Manifold can be additionally provided with a control, for controlling the radial expansion of the distal segment of the catheter. Control may take any of a variety of forms depending upon the mechanical structure of the support. In the illustrated embodiment, control comprises a slider switch, which is mechanically axially moveably linked to the distal support (discussed below) such that proximal retraction of the slider switch produces a proximal movement of the support. This allows the unsupported distal section to assume its low profile configuration. Distal axial advancement of the slider switch produces a distal axial advance of the support. In the distal position, the support advances the distal segment from the reduced diameter, to the enlarged diameter. In the enlarged configuration, the support maintains patency of a central lumen extending through the distal segment to accommodate aspiration as will be discussed below.

Any of a variety of controls may be utilized, including switches, levers, rotatable knobs, pull/push wires, and others, which will be apparent to those of skill in the art in view of the disclosure herein.

Guide catheters of the present invention, which are adapted for intracranial applications, generally have a total length in the range of from 60 cm to 250 cm, usually from about 135 cm to about 175 cm. The length of the proximal segment will typically be from 20 cm to 220 cm, more typically from 100 cm to about 120 cm. The length of the distal segment will typically be in the range from 2 cm to about 50 cm, usually from about 5 cm to about 30 cm. The proximal and distal body segments 104, 110 may be joined to each other, i.e. at a transition 108. The body segments may be joined in any of a variety of conventional manners, such as heat fusion, adhesive bonding, co-extrusion, or the like. In the exemplary embodiment, the two body segments 104, 110 will be formed separately and thereafter welded together. The polymeric surround structures 102 and 112 can be fused by the application of heat with a removable mandrel extending through each lumen, which crosses the transition to maintain patency. A length of outer shrink-wrap tubing may be used to add structural integrity by spanning the transition.

The catheters of the present invention may be composed of any of a variety of biologically compatible polymeric resins having suitable characteristics when formed into the tubular catheter body segments. Exemplary materials include polyvinyl chloride, polyethers, Hytrel, Pebax, polypropylene, polyamides, polyethylenes, polyurethanes, copolymers thereof, and the like. In certain embodiments, in which the distal segment dilates (stretches) radially rather than unfolds, the distal segment may be formed from more elastic materials, such as latex rubber, silicone rubber, and blends thereof. In one embodiment, both the proximal body segment 33 and distal body segment will comprise a polyvinyl chloride (PVC), with the proximal body segment being formed from a relatively rigid PVC and the distal body segment being formed from a relatively flexible, supple PVC. Optionally, the proximal body segment may be reinforced with a metal or polymeric braid or other conventional reinforcing layer.

The proximal body segment will exhibit sufficient column strength to permit axial positioning of the catheter through a guide catheter at least a portion of with the distal body segment extending into the patient's vasculature. The proximal body segment may have shore hardness in the range from 50 D to 100 D, often being about 70 D to 80 D. Usually, the proximal shaft will have a flexural modulus from 20,000 psi to 1,000,000 psi, preferably from 100,000 psi to 600,000 psi. The distal body segment will be sufficiently flexible and supple so that it may navigate the patient's distal vasculature. In highly flexible embodiments, the shore hardness of the distal body segment may be in the range of from about 20 A to about 100 A, and the flexural modulus for the distal segment may be from about 50 psi to about 15,000 psi.

The catheter body may further comprise other components, such as radiopaque fillers; colorants; reinforcing materials; reinforcement layers, such as braids and helical reinforcement elements; or the like. In particular, the proximal body segment may be reinforced in order to enhance its column strength and torqueability while preferably limiting its wall thickness and outside diameter.

Usually, radiopaque markers will be provided at least at the distal end and the transition region of the outer catheter or the translation dilator 100. Other radiopaque markers may be provided elsewhere, such as on the support coil, if it is not already radiopaque. One radiopaque marker comprises a metal band, which is fully recessed within the distal end of the proximal body segment. Suitable marker bands can be produced from a variety of materials, including platinum, gold, and tungsten/rhenium alloy. Preferably, the radiopaque metal band will be recessed in an annular channel formed at the distal end of the proximal body segment.

The proximal section of tubular body may be produced in accordance with any of a variety of known techniques for manufacturing interventional catheter bodies, such as by extrusion of appropriate biocompatible polymeric materials. Alternatively, at least a proximal portion or all of the length of tubular body may comprise a polymeric or metal spring coil, solid walled hypodermic needle tubing, or braided reinforced wall, as is known in the microcatheter arts.

In many applications, the proximal section of tubular body is provided with an approximately circular cross-sectional configuration having an external diameter within the range of from about 0.025 inches to about 0.065 inches. In accordance with one embodiment of the invention, the proximal section 33 of tubular body has an external diameter of about 0.042 inches (3.2 f) throughout most of its length. Alternatively, a generally oval or triangular cross-sectional configuration can also be used, as well as other noncircular configurations, depending upon the method of manufacture, number and arrangement of internal lumens and the intended use.

In a catheter intended for peripheral vascular applications, the proximal section of body will typically have an outside diameter within the range of from about 0.039 inches to about 0.065 inches. In coronary vascular applications, the proximal section of body will typically have an outside diameter within the range of from about 0.025 inches to about 0.045 inches. The illustrated construction of distal section permits lower external cross-sections in the collapsed configuration, as low as 0.028 inches or 0.025 inches or 0.022 inches or lower as may be desired for remote coronary or intracranial applications.

Diameters outside of the preferred ranges may also be used, provided that the functional consequences of the diameter are acceptable for the intended purpose of the catheter. For example, the lower limit of the diameter for any portion of tubular body in a given application will be a function of the number of fluid or other functional lumen contained in the catheter, together with the acceptable minimum aspiration flow rate and collapse resistance.

Tubular body must have sufficient structural integrity (e.g., column strength or “pushability”) to permit the catheter to be advanced to distal locations without buckling or undesirable bending of the tubular body. The ability of the body to transmit torque may also be desirable, such as to avoid kinking upon rotation, to assist in steering. The tubular body, and particularly the distal section, may be provided with any of a variety of torque and/or column strength enhancing structures. For example, axially extending stiffening wires, spiral wrapped support layers, braided or woven reinforcement filaments may be built into or layered on the tubular body.

In many applications, the proximal section will not be required to traverse particularly low profile or tortuous arteries. For coronary vascular applications, for example, the proximal section will be mostly or entirely within the relatively large diameter guide catheter. The transition can be located on the catheter shaft to correspond approximately with the distal end of the guide catheter when the balloon and/or distal end is at the treatment site. Viewed the other way, the length of the distal section is preferably at least as long as the distance from the ostium of the relevant coronary artery to the treatment site. In most applications, the transition will be at least about 3 cm, preferably at least about 5 cm and alternatively as much as about 10 cm but often not more than about 20 cm from the distal end of the catheter. Distances as much as 30 cm to 50 cm or greater between the transition and distal end of the catheter may also be desirable in some applications.

For certain other applications, such as intracranial catheterizations, the distal section is preferably at least about 5 cm long and small enough in diameter to pass through vessels as low as 3 mm or 2 mm or lower. Catheters for this application may have a proximal section length of between about 60 cm to about 150 cm and a distal section length of between about 5 cm to about 15 cm, and the distal section is able to track a tortuous path of at least about 5 cm through vessels of less than about 3 mm lumen ID.

The distal section, may be manufactured as an extrusion. In one method of manufacture, the extrusion is formed from a medium to high melt index polyethylene or other polymer having an outside diameter of greater than the diameter of the desired finished product. The raw extrusion can thereafter be drawn down to the desired diameter, in accordance with known processing techniques. The draw down pull speed can be varied such as along a proximal portion of the extrusion to produce a taper to a larger proximal diameter. This permits a smooth transition 32 from the relatively smaller outside diameter distal section to the typically larger outside diameter of proximal section. High melt index materials allow the production of a greater number of different diameter draw downs by adjusting pull speed and other process parameters, for a given set of tooling, as will be appreciated by those of skill in the art. The distal end 14 can be further reduced in diameter by an additional draw down step if desired.

Referring to FIG. 1, the axially moveable support 100 may be provided in the form of an elongate flexible tube. A proximal section 116 of tubular element 100 is provided with a spiral cut reinforcement 104, to retain radial strength but provide lateral flexibility. The spiral 110 within the distal section 118 generally has a length within the range of from about 1 centimeter to 30 centimeters, preferably within a range of about 5 centimeters to about 20 centimeters, and, in a particular embodiment, extends for approximately 15 centimeters in length. The spiral cut 110 generally has a pitch within the range of from about 0.01 inches to about 0.125 inches, and in one embodiment, has a 0.06 pitch. In another embodiment, the distal section comprises a first spiral cut section having a length of about 5 cm and a pitch of about 0.06, and a second, distal section having a length of about 5 cm and a pitch of about 0.030.

Preferably, the spiral cut extends completely through the wall of the distal reinforcing element 110 to produce a helical or coiled configuration. The precise pitch of the spiral cut and axial spacing of adjacent windings can be varied widely while still accomplishing the purposes of the present invention, and can be optimized for any particular application in view of the disclosure herein. In a preferred embodiment, the distal reinforcing element 110 is a coil of flat wire wound around a mandrel using a coil winder.

For example, polytetrafluoroethylene tubing, such as that suitable for tubular element 30, can be commercially obtained from Zeus, in Orangeburg, S.C. The distal section 32 can be provided with a spiral cut, such as by any of a variety of techniques that can be devised by those of skill in the art. In accordance with one technique, the PTFE or other tubing is placed onto a mandrel. The mandrel is attached to a machine with a predetermined screw thread. A cutting element such as a razor blade or other sharp instrument is placed across the tubing and the machine is activated to rotate the mandrel. As rotation of the machine (screw thread) occurs, the mandrel moves axially and rotationally causing the tubing to be cut in a spiral manner by the cutting implement. The machine can be set up to cut either a right or left hand spiral. The machine can also be set to cut continuous or variable pitch spirals, or multi-zone spiral sections in which each zone has a unique pitch. A metal spring coil 34 can be wrapped about a suitably sized rotating mandrel as is known in the art, with the distal open wound section 36 formed by stretching.

The distal reinforcement 110 may alternatively comprise a wire spring, extending throughout the length of the distal segment 118 or entire catheter 100. A distal section of the coil spring 110 can be stretched axially to produce an open wound configuration, such that the axial space between adjacent windings of the coil may be within the range of from about 0.05 mm to about 1 mm or greater. The proximal portion of the distal section coil spring 110 can be generally bottomed out (not illustrated), such that adjacent windings of the coil are in contact with one another. This provides column strength, to allow distal advancement within the catheter, while retaining lateral flexibility. Alternatively, the coil spring 110 can be open wound with, e.g., 0.01 mm to 1 mm spacing for the entire length.

A variety of materials can be used to construct the coil spring 110, such as stainless steel, nitinol, cobalt-nickel alloy, platinum, platinum alloy, nickel, or titanium alloys. Coil spring 110 can be produced from any of a variety of stock forms, such as round cross-sectional wire, square or other rectangular wire, or polymeric materials as are known in the art. In one embodiment, coil spring 110 is wound from a flat wire made from stainless steel and having cross-sectional dimensions of about 0.001 by about 0.010 inches, about 0.002 by about 0.008 inches, or the like.

Access for the catheter of the present invention can be achieved using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. An incision can also be made on right carotid artery or left carotid artery in emergency situations.

The length of the catheter for those access sites to reach the brain will generally be between 20 to 100 centimeters, preferably approximately between 30 and 60 centimeters. The inner diameter of the catheter may be between 0.2 and 0.6 centimeters, or smaller. The foregoing ranges are set forth solely for the purpose of illustrating typical device dimensions. The actual dimensions of a device constructed according to the principles of the present invention may obviously vary outside of the listed ranges without departing from those basic principles.

The construction disclosed herein is suitable for guide catheter as a stand-alone device. The same construction can be used as the translation dilator or axially movable dilator within a guide catheter comprising a distal, expandable region. This construction results in a highly flexible device having high column strength, torqueability, kink-resistance, and tensile strength.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following objects of the invention.

1. A guide catheter device comprising:
a non-diametrically expandable proximal tubing segment comprising an axially elongate tube having a proximal end, a distal end, and a lumen extending therethrough, the flexibility of said proximal tubing segment being greater at its distal end than at its proximal end;
a diametrically expandable distal segment affixed to the distal end of the proximal tubing segment, the distal segment responsive to expand diametrically upon axial movement of a hollow central dilator into or out of the distal segment; and
a hollow central dilator comprising a composite structure of an inner liner, an outer polymer layer, and a reinforcement;
wherein the hollow central dilator comprises at least two regions of flexibility determined, in part, or in whole, by the construction of the reinforcement in each of the two regions of flexibility.

2. A device according to claim 1 further comprising a hub attached to the proximal end of the proximal tubing segment.

3. A device according to claim 2 wherein the hub comprises at least one hemostasis valve and at least one access port.

4. A system useable for performing a therapeutic or diagnostic task at a location within the body of a human or animal subject, said system comprising a) catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion and b) a working device that is advanceable though the lumen of the catheter and out of its distal opening at least when the distal portion of the catheter is in is second configuration, said working device being useable to perform the therapeutic or diagnostic task.

5. A system according to claim 4 wherein the working device is selected from: i) devices for removing thrombus or other obstructive matter from body lumens, ii) flow restoration devices useable to facilitate flow of a fluid though or around an obstruction within a body lumen and iii) devices for deploying or delivering occlusion coils, embolic devices or other implants.

6. A method for performing a therapeutic or diagnostic task at a location within the body of a human or animal subject, such method comprising the steps of: a) inserting into the subject's body a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the distal end opening in a desired body lumen while the distal portion of the catheter is in its first configuration; c) causing the distal portion of the catheter to transition to its second configuration; d) advancing a working device though the lumen of the catheter and out of its distal opening; and, e) using the working device to perform the therapeutic or diagnostic task.

7. A method according to claim 6 wherein the working device is selected from: devices for removing thrombus or other obstructive matter from body lumens, flow restoration devices useable to restore blood flow though an obstructed body lumen and devices for delivering occlusion coils, embolic devices or other implants.

8. A method for removing obstructive matter from a body lumen, said method comprising the steps of: a) inserting a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the catheter, while in the first configuration, such that its distal end opening is within a body lumen; c) causing the catheter to transition from the first configuration to the second configuration; d) moving obstructive matter through the distal end opening and into the lumen of the catheter; and e) removing the catheter along with the obstructive matter that has been moved into the lumen of the catheter.

9. A method according to claim 8 wherein negative pressure is applied through a lumen of the catheter to aspirate obstructive matter through the distal end opening and into the catheter.

10. A method according to claim 8 wherein Steps D and E comprise advancing an obstructive matter moving device from the catheter and using the obstructive matter-moving device to move obstructive matter through the distal end opening and into the lumen of the catheter.

11. A method according to claim 10 wherein the obstructive matter moving device comprises an embolectomy device.

12. A method for increasing flow of a body fluid through an obstructed body lumen, such method comprising the steps of: a) inserting a catheter that has a proximal portion, a distal portion, a lumen and a distal end opening, said catheter being transitionable from a first configuration wherein the distal portion has a first outer diameter that is smaller than the outer diameter of the proximal portion and a second configuration wherein the distal portion is expanded to a second outer diameter that is larger than the first outer diameter and no larger than the outer diameter of the proximal portion; b) positioning the catheter, while in the first configuration, such that its distal end opening is within a body lumen; c) causing the catheter to transition from the first configuration to the second configuration; and d) using the catheter to deliver a treatment that restores or improving flow of a body fluid through an obstructed body lumen.

13. A method according to claim 12 wherein Step D comprises delivering a therapeutic substance of a type and in an amount that is effective to improve flow of body fluid through the body lumen.

14. A method according to claim 13 wherein the therapeutic substance comprises a thrombolytic agent.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)
